(19) United States
(12) Patent Application Publication
(10) Pub. No.: US 2026/0148815 A1
(43) Pub. Date: May 28, 2026

(54) INFORMATION PROCESSING DEVICE, MEDICAL DOCUMENT CONFIRMATION SUPPORT METHOD, AND NON-TRANSITORY RECORDING MEDIUM

(71) Applicant: NEC Corporation, Tokyo (JP)

(72) Inventors: Fumihiro TANIGUCHI, Tokyo (JP); Takuma Sato, Tokyo (JP)

(73) Assignee: NEC Corporation, Tokyo (JP)

(21) Appl. No.: 19/386,488

(22) Filed: Nov. 12, 2025

(30) Foreign Application Priority Data
Nov. 27, 2024 (JP) 2024-206307

(51) Int. Cl. G16H 10/60

(52) U.S. Cl. CPC G16H 10/60

(57) ABSTRACT

An information processing device includes an identification unit for identifying a description of relevant contents between original data indicating a medical care history of a patient and a medical document generated by using the original data, and a detection unit for detecting a description in which the description of relevant contents has not been identified by the identification unit. According to this information processing device, it is also possible to optimize the confirmation work of the medical document.

### INCORPORATION BY REFERENCE

This application is based upon and claims the benefit of priority from Japanese patent application No. 2024-206307, filed on November 27, 2024, the disclosure of which is incorporated herein in its entirety by reference.

### TECHNICAL FIELD

The present disclosure relates to an information processing device, a medical document confirmation support method, and a non-transitory recording medium.

### BACKGROUND ART

In addition to medical practice such as medical examination and treatment, many doctors perform work of creating medical documents such as a report of a treatment progress, a medical introduction letter, and an insurance medical certificate. Examples of a technique for reducing a burden on a doctor in such work include an information processing device described in JP 2023-059686 A below. The information processing device derives record information to be recorded in a record item of a medical document based on patient information, and records the derived record information in the record item to generate medical document data. By using this information processing device, it is possible to reduce the burden of the work of creating a medical document.

### SUMMARY

However, the medical document data generated by the information processing device does not necessarily have appropriate contents. Therefore, even in the case of using the information processing device described in JP 2023-059686 A, the doctor is not released from the work of comparing the generated medical document data with the original medical record or the like and confirming whether there is no inconsistency in the contents, whether there is no omission or the like.

As described above, the information processing device described in JP 2023-059686 A has room for improvement in that it cannot support confirmation work of a generated medical document. The confirmation work of the medical document is a burden not only in a case where the information processing device is caused to generate the medical document but also in a case where the doctor or the like creates the medical document by himself/herself. An example object of the present disclosure is to provide a technique for facilitating the confirmation work of a medical document.

An information processing device according to an example aspect of the present disclosure includes an identification means for identifying, for original data indicating a medical care history of a patient and a medical document of the patient generated using the original data, a description of relevant contents between the original data and the medical document, and a detection means for detecting a description in which the description of relevant contents has not been identified by the identification means.

Another information processing device according to an example aspect of the present disclosure includes a matching determination means for determining whether contents of a description of a generated medical document match contents of a description of original data, which is data from which the medical document is generated, by using a language model obtained by machine learning of a natural language, and an output control means for outputting a determination result of the matching determination means.

In a medical document confirmation support method according to an example aspect of the present disclosure, at least one processor executes an identification process of identifying, for original data indicating a medical care history of a patient and a medical document of the patient generated by using the original data, a description of relevant contents between the original data and the medical document, and a detection process of detecting a description in which a description of relevant contents has not been identified in the identification process.

A medical document confirmation support program according to an example aspect of the present disclosure causes a computer to function as an identification means for identifying, for original data indicating a medical care history of a patient and a medical document of the patient generated using the original data, a description of relevant contents between the original data and the medical document, and a detection means for detecting a description in which the description of relevant contents has not been identified by the identification means.

According to an example aspect of the present disclosure, there is an exemplary effect that a confirmation work of a medical document can be facilitated.

### BRIEF DESCRIPTION OF DRAWINGS

FIG. 1 is a block diagram illustrating a configuration of an information processing device according to the present disclosure;

FIG. 2 is a flowchart illustrating a flow of a confirmation support method according to the present disclosure;

FIG. 3 is a block diagram illustrating a configuration of another information processing device according to the present disclosure;

FIG. 4 is a diagram illustrating an example of processing performed by the information processing device illustrated in FIG. 3;

FIG. 5 is a diagram for explaining a method of identifying a description of relevant contents using a feature information generation model and a similarity estimation model;

FIG. 6 is a diagram for explaining a method of determining consistency using a language model;

FIG. 7 is a diagram illustrating an example of a display screen displayed by an output control unit;

FIG. 8 is a diagram illustrating another example of the display screen displayed by the output control unit;

FIG. 9 is a flowchart illustrating a flow of processing executed by the information processing device illustrated in FIG. 3;

FIG. 10 is a flowchart illustrating details of the processing in S13 in FIG. 9;

FIG. 11 is a block diagram illustrating a configuration of an information processing device according to a reference example; and

FIG. 12 is a block diagram illustrating a configuration of a computer that functions as an information processing device according to the present disclosure.

### EXAMPLE EMBODIMENT

Hereinafter, example embodiments of the present invention will be described. However, the present invention is not limited to the following exemplary example embodiments, and various modifications can be made within a scope described in the claims. For example, example embodiments obtained by appropriately combining technologies (some or all of things or methods) adopted in the following exemplary example embodiments can also be included in the scope of the present invention. Example embodiments obtained by appropriately omitting some of the technologies adopted in the following exemplary example embodiments can also be included in the scope of the present invention. Effects mentioned in the following exemplary example embodiments are examples of effects expected in the exemplary example embodiments, and do not define extension of the present invention. That is, example embodiments that do not achieve the effects mentioned in the following exemplary example embodiments can also be included in the scope of the present invention.

### First exemplary example embodiment

A first exemplary example embodiment that is an example of the example embodiments of the present invention will be described in detail with reference to the drawings. The present exemplary example embodiment is a basic form of each exemplary example embodiment to be described below. An application range of each technology adopted in the present exemplary example embodiment is not limited to the present exemplary example embodiment. That is, each technology adopted in the present exemplary example embodiment can also be adopted in another exemplary example embodiment included in the present disclosure within a range in which no particular technical problem occurs. Each technique illustrated in the drawings referred to for describing the present exemplary example embodiment can also be adopted in the other exemplary example embodiments included in the present disclosure within a range in which no particular technical problem occurs.

### Configuration of information processing device 1

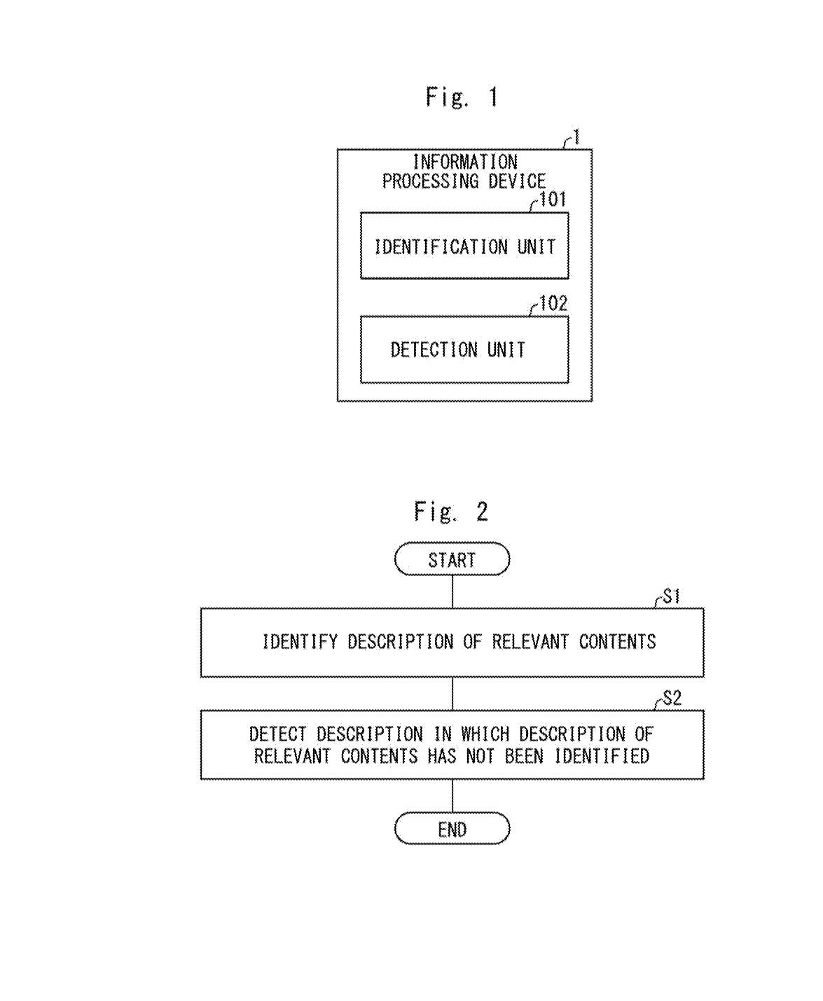

A configuration of an information processing device 1 according to the present exemplary example embodiment will be described with reference to FIG. 1. FIG. 1 is a block diagram illustrating a configuration of the information processing device 1. As illustrated in FIG. 1, the information processing device 1 includes an identification unit 101 and a detection unit 102.

The identification unit 101 identifies, for original data indicating a medical care history of a patient and a medical document of the patient generated using the original data, descriptions of relevant contents between the original data and the medical document.

The “original data” indicates a medical care history of a patient, and may include information necessary for generating a desired medical document. For example, any one or more of a medical record (an electronic medical record may be used, or a paper medical record may be imaged and subjected to character recognition) of the patient, data indicating a result of examination or diagnosis received by the patient, and data indicating a medicine prescribed to the patient may be used as the original data. The original data may be data indicating a medical care history of a patient in natural language.

The “medical document” indicates the content of medical treatment performed on a subject patient in a predetermined format. For example, the medical document may be various medical certificates such as an insurance medical certificate, a treatment progress report, a medical introduction letter, or the like. The medical document may be automatically generated by the information processing device 1 or another device, may be manually generated by a doctor or the like, or may be partially automatically generated and another portion may be manually generated. The identification unit 101 performs the above-described processing of identifying a description of relevant contents for the medical document and the original data which are electronic data.

The above “description of relevant contents” may be any description having some relevance to the contents. Depending on the method of identification to be applied, what relevant descriptions are identified for the content may vary. For example, in a case where descriptions having similar contents are identified as “description of relevant contents”, if both the original data and the medical document include a sentence indicating that the patient's chronic disease is diabetes, the identification unit 101 identifies these sentences as a description of relevant contents.

The detection unit 102 detects a description in which the description of relevant contents has not been identified by the identification unit 101. By using the detection result of the detection unit 102, the confirmation work of the medical document can be facilitated.

For example, it is assumed that the original data includes a sentence indicating that a patient's chronic disease is diabetes, but a description of contents relevant to the sentence has not been identified from the medical document. In this case, the detection unit 102 detects the sentence as a description in which the description of relevant contents has not been identified by the identification unit 101. In this case, the detected description may be a description that is not reflected in the medical document. That is, in this case, by using the detection result of the detection unit 102, it is possible to easily find a description that is not reflected in the medical document.

On the contrary, it is assumed that the medical document includes a sentence indicating that the patient's chronic disease is diabetes, but the description of the contents relevant to the sentence is not identified from the original data. Also in this case, the detection unit 102 detects the sentence as a description in which the description of relevant contents has not been identified by the identification unit 101. In this case, the detected description may have been erroneously written in the medical document. That is, in this case, it is possible to easily find a description erroneously written in the medical document by using the detection result of the detection unit 102.

The description in which the description of relevant contents has not been identified by the identification unit 101 may include, for example, a description in which the content has been modified to such an extent that the relevant description cannot be identified, a description that does not need to be described in a medical document such as a greeting sentence or an acknowledgement, and the like, in addition to the description that has been erroneously written and the description that has not been reflected. Therefore, the detection unit 102 may detect the remaining description from the description in which the description of relevant contents has not been identified by the identification unit 101 except for at least one of the description in which the content is modified and the description unnecessary to be described in the medical document. The description whose content has been modified can be detected by, for example, the method described in the second exemplary example embodiment described later. Description unnecessary to be described in the medical document can be detected, for example, by listing such description in advance.

As described above, in the information processing device 1 according to the present exemplary example embodiment, a configuration is adopted in which original data indicating a medical care history of a patient and a medical document generated using the original data are targeted, and the information processing device 1 includes the identification unit 101 that identifies a description of relevant contents between the original data and the medical document, and the detection unit 102 that detects a description in which a description of relevant contents has not been identified by the identification unit 101.

As described above, the detection result of the detection unit 102 can be used to find a description that has not been reflected in the medical document or a description that has been erroneously written in the medical document. Therefore, according to the information processing device 1, it is possible to obtain an effect of facilitating the confirmation work of the medical document. According to the information processing device 1, it is also possible to optimize the confirmation work of the medical document.

How to use the detection result of the detection unit 102 in the medical document confirmation work is arbitrary. For example, the information processing device 1 may present the detection result of the detection unit 102 to the user of the information processing device 1 (for example, a final checker of a medical document such as a doctor). As a result, the user can efficiently confirm the appropriateness/inappropriateness of the content of the medical document with reference to the detection result of the detection unit 102, and perform correction and the like as necessary. The detection result of the detection unit 102 can also be used for further analysis of a medical document, improvement of processing of generating a medical document, and the like. Even in a case where these usage modes are applied, it finally leads to the facilitation of the medical document confirmation work.

### Medical document confirmation support program

The functions of the information processing device 1 described above can also be achieved by a program. A medical document confirmation support program according to the present exemplary example embodiment causes a computer to function as an identification means for identifying, for original data indicating a medical care history of a patient and a medical document of the patient generated using the original data, a description of relevant contents between the original data and the medical document, and a detection means for detecting a description of which relevant contents have not been identified by the identification means. According to this confirmation support program, it is possible to obtain an effect of facilitating the confirmation work of the medical document.

### Flow of medical document confirmation support method

A flow of a medical document confirmation support method according to the present exemplary example embodiment will be described with reference to FIG. 2. FIG. 2 is a flowchart illustrating a flow of the medical document confirmation support method. An executing entity of each step in this confirmation support method may be a processor included in the information processing device 1, may be a processor included in another device, or an executing entity of each step may be a processor provided in each of different devices.

In S1 (identification process), at least one processor targets original data indicating a medical care history of a patient and a medical document of the patient generated using the original data, and identifies a description of relevant contents between the original data and the medical document.

In S2 (detection process), at least one processor detects a description in which a description of relevant contents has not been identified in the processing of S1 from at least one of the original data and the medical document. Accordingly, the processing of FIG. 2 ends. If reflection omission, incorporation of erroneous information, or the like is not performed in the medical document, the description is not detected in S2.

As described above, the medical document confirmation support method according to the present exemplary example embodiment employs a configuration in which at least one processor executes, for original data indicating a medical care history of a patient and a medical document of the patient generated using the original data, an identification process of identifying a description of relevant contents between the original data and the medical document, and a detection process of detecting a description in which a description of relevant contents has not been identified in the identification process. According to this confirmation support method, it is possible to obtain an effect of facilitating the confirmation work of the medical document.

### Second exemplary example embodiment

A second exemplary example embodiment that is an example of the example embodiments of the present invention will be described in detail with reference to the drawings. Components that have the same functions as the components described in the above-described exemplary example embodiment are denoted by the same reference signs, and will not be described as appropriate. An application range of each technology adopted in the present exemplary example embodiment is not limited to the present exemplary example embodiment. That is, each technology adopted in the present exemplary example embodiment can also be adopted in another exemplary example embodiment included in the present disclosure within a range in which no particular technical problem occurs. Each technique illustrated in each of the drawings referred to for describing the present exemplary example embodiment can be employed in the other exemplary example embodiments included in the present disclosure within a range in which no particular technical problem occurs.

### Configuration of information processing device 1A

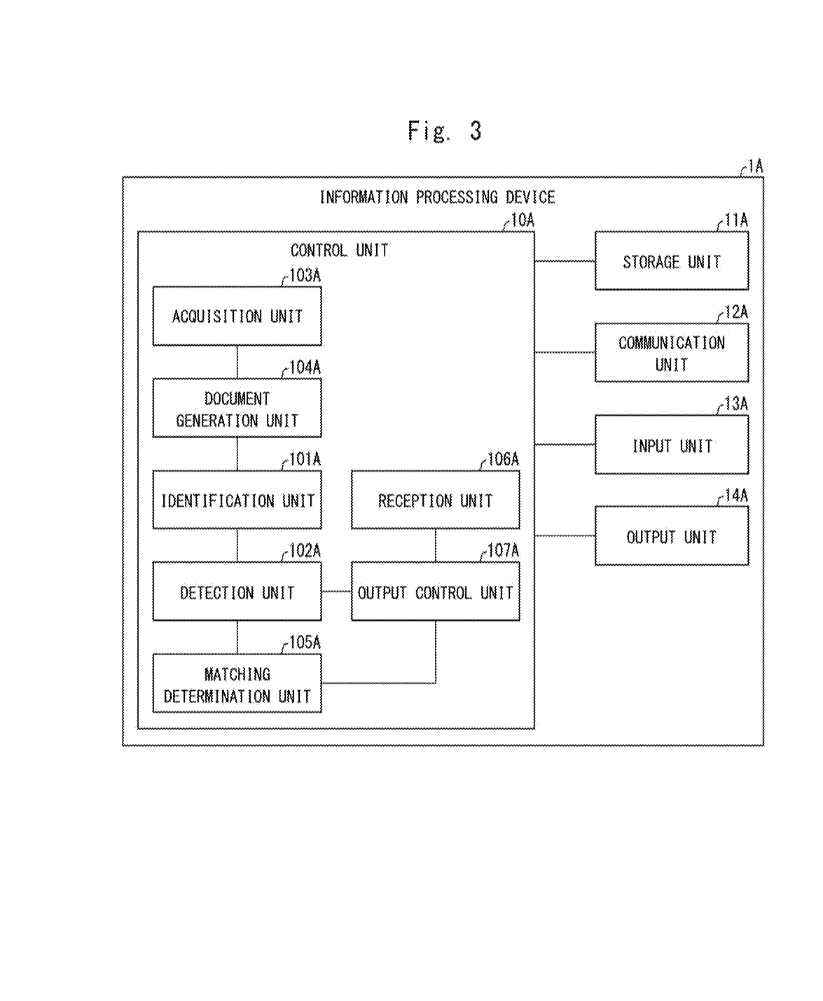

A configuration of an information processing device 1A according to the present exemplary example embodiment will be described with reference to FIG. 3. FIG. 3 is a block diagram illustrating the configuration of the information processing device 1A. The information processing device 1A is a device having a function of supporting confirmation work of contents of a medical document. The information processing device 1A may be a local device used by individual users, or may be a server that provides a medical document confirmation support service to a plurality of users.

As illustrated, the information processing device 1A includes a control unit 10A that integrally controls units of the information processing device 1A, and a storage unit 11A that stores various types of data to be used by the information processing device 1A. The information processing device 1A includes a communication unit 12A for the information processing device 1A to communicate with another device, an input unit 13A for accepting an input to the information processing device 1A, and an output unit 14A for the information processing device 1A to output data. The control unit 10A includes an identification unit 101A, a detection unit 102A, an acquisition unit 103A, a document generation unit 104A, a matching determination unit 105A, a reception unit 106A, and an output control unit 107A.

Similarly to the identification unit 101 of the first exemplary example embodiment, the identification unit 101A identifies a description of relevant contents between the original data and the medical document for the original data indicating a medical care history of a patient and the medical document of the patient generated using the original data. Although details will be described later, two machine-learned models of a feature information generation model M1 and a similarity estimation model M2 are used to identify the description of relevant contents.

Similarly to the detection unit 102 of the first exemplary example embodiment, the detection unit 102A detects a description in which the description of relevant contents has not been identified by the identification unit 101A. For example, in a case where the original data is set as the detection target, the detection unit 102A may detect the remaining description from the description included in the original data except for the description identified by the identification unit 101A. This makes it possible to detect a description suspected of omission of reflection in the medical document. Similarly, the detection unit 102A may detect the remaining description from the description of the medical document except for the description identified by the identification unit 101A. The description detected in this way is a description suspected of being erroneously written in the medical document.

The acquisition unit 103A acquires various types of data related to medical document confirmation support. For example, the acquisition unit 103A acquires original data that is a source of a medical document. The data acquisition method is arbitrary, and for example, the acquisition unit 103A may acquire data from an external device (for example, a terminal device or the like used by the user) via the communication unit 12A, or may acquire data input to the information processing device 1A via the input unit 13A.

The document generation unit 104A generates a medical document from the original data acquired by the acquisition unit 103A. A method of generating the medical document is arbitrary. For example, the document generation unit 104A may generate a medical document by detecting each item to be filled in the generated medical document from the original data and inputting each detected item to the template of the medical document. A language model obtained by machine learning of natural language may be used to generate the medical document.

Here, machine learning on natural language more specifically means learning of the arrangement of components (words and the like) in a sentence in a natural language and the arrangement of sentences in a text. Examples of the language model trained on natural language include bidirectional encoder representations from transformers (BERT), robustly optimized BERT approach (RoBERTa), efficiently learning an encoder that classifies token replacements accurately (ELECTRA), and the like.

The matching determination unit 105A determines whether the contents of the description match between the original data and the medical document by using a language model obtained by machine learning of natural language. As described above, the language model in which the natural language is machine-learned is a machine-learned model in which the arrangement of the components in the sentence of the natural language and the arrangement of sentences in the document are learned. Hereinafter, the language model used by the matching determination unit 105A is referred to as a language model M3.

In the present exemplary example embodiment, an example in which the language model M3 is a model that accepts an input of a prompt in a text format described in a natural language and outputs an answer in the natural language will be described. However, the language model M3 may be a model capable of accepting input of data in a format other than text data such as an image. As a result, it is possible to facilitate confirmation work of a medical document including data in a format other than text data.

The language model M3 may be a general-purpose language model that can be used for applications other than inference of consistency in contents of the description, or may be a general-purpose language model finely tuned for inference of consistency in contents of the description.

In the present exemplary example embodiment, an example in which the medical document generated by the document generation unit 104A is a target of detection by the detection unit 102A and determination of consistency by the matching determination unit 105A will be described. However, the medical document to be determined for consistency may be generated using original data. That is, a generation subject and a generation method of the medical document to be a target of the detection by the detection unit 102A and the consistency determination by the matching determination unit 105A are arbitrary.

The reception unit 106A receives various operations related to the medical document confirmation support. For example, the reception unit 106A receives an operation of designating a part of a description included in a medical document or original data. Any method of receiving the operation is applicable. For example, the reception unit 106A may receive an operation via the input unit 13A, or may receive an operation from another device via the communication unit 12A.

The output control unit 107A presents various types of information regarding the medical document confirmation support. For example, as described above, the output control unit 107A displays the medical document and the original data. For example, the output control unit 107A displays the detection result of the detection unit 102A and the determination result of the matching determination unit 105A.

In a case where the output unit 14A has a function of displaying and outputting an image, the output control unit 107A may cause the output unit 14A to display the data described above. The output control unit 107A may display the data described above on a display device (for example, a display device included in a terminal device used by the user) outside the information processing device 1A via the communication unit 12A.

A method of presenting information is arbitrary and is not limited to display. For example, the output control unit 107A can present information in any form such as display, printing, voice, or a combination thereof. That is, the output control unit 107A may output the information such as the detection result of the detection unit 102A and the determination result of the matching determination unit 105A to any device and in any mode.

As described above, the information processing device 1A includes the identification unit 101A that identifies, for original data indicating a medical care history of a patient and a medical document generated by using the original data, a description of relevant contents between the original data and the medical document, and the detection unit 102A that detects a description in which the description of relevant contents has not been identified by the identification unit 101A. Therefore, according to the information processing device 1A, similarly to the information processing device 1, it is possible to obtain an effect that it is possible to facilitate the confirmation work of the medical document, more specifically, the work of confirming the presence or absence of a description that has not been reflected in the medical document and/or a description that has been erroneously written in the medical document.

As described above, the information processing device 1A includes the matching determination unit 105A that determines, for a set of descriptions identified by the identification unit 101A, whether the contents of the descriptions match using the language model M3 obtained by machine learning of a natural language. As a result, in addition to the effect obtained by the information processing device 1, it is possible to easily check whether there is a description not matching with the original data in the medical document.

The determination by the matching determination unit 105A can be omitted. In a case where the determination by the matching determination unit 105A is omitted, the output control unit 107A may output the detection result of the detection unit 102A. As a result, the user of the information processing device 1A can efficiently check whether there is a description that has not been reflected in the medical document and/or a description that has been erroneously written in the medical document with reference to the output detection result.

As described above, the information processing device 1A includes the matching determination unit 105A that determines whether the content of the description of the generated medical document matches the content of the description of the original data, which is data from which the medical document is generated, by using a language model obtained by machine learning of a natural language, and the output control unit 107A that causes a determination result of the matching determination unit 105A to be output. Therefore, it is possible to facilitate the confirmation work of the medical document, more specifically, the presence or absence of a description not matching with the original data in the medical document.

### Specific example of processing

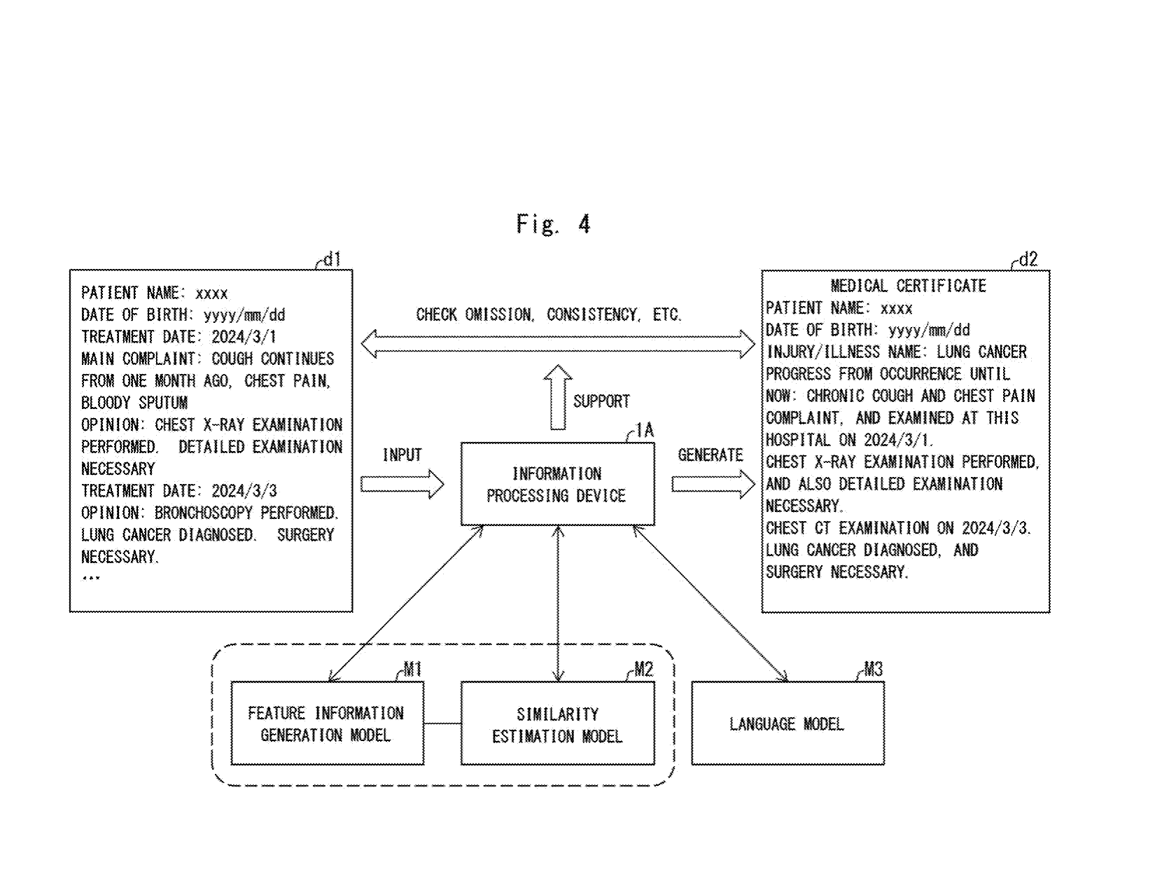

An example of processing executed by the information processing device 1A will be described with reference to FIG. 4. FIG. 4 is a diagram illustrating an example of the processing executed by the information processing device 1A. In the example of FIG. 4, original data d1 is input to the information processing device 1A. The original data d1 is data in which a medical care history of a patient whose name is “XXXX” and date of birth is “yyyy/mm/dd” is indicated in natural language. The input original data d1 is acquired by the acquisition unit 103A included in the information processing device 1A.

In the example of FIG. 4, the document generation unit 104A generates a medical document (specifically, a medical certificate) d2 from the acquired original data d1. Then, the information processing device 1A supports the work of confirming the presence or absence of omission and consistency with the original data d1 for the generated medical document d2. This support is mainly implemented by the identification unit 101A, the detection unit 102A, and the matching determination unit 105A. The feature information generation model M1, the similarity estimation model M2, and the language model M3 illustrated in FIG. 4 are used for this support.

The language model M3 may also be used to generate the medical document d2. These models may be stored in the storage unit 11A or the like of the information processing device 1A, or may be stored in a server or the like outside the information processing device 1A. In the latter case, the information processing device 1A uses each model via a server or the like that stores the model.

The feature information generation model M1 is a machine-learned model that is machine-learned so as to generate feature information indicating a feature of an input sentence. For example, Bi-Encoder or the like that generates an embedding vector indicating a feature of an input sentence can be used as the feature information generation model M1.

The similarity estimation model M2 is a machine-learned model that is machine-learned so as to output the similarity of contents of the sentence with respect to a set of the input sentences. Such a model can be generated by machine learning using training data in which a correct answer label indicating that the sentences are similar to a set of similar sentences is associated with each other and training data in which a correct answer label indicating that the sentences are dissimilar to a set of dissimilar sentences is associated with each other. For example, Cross-Encoder or the like can be used as the similarity estimation model M2.

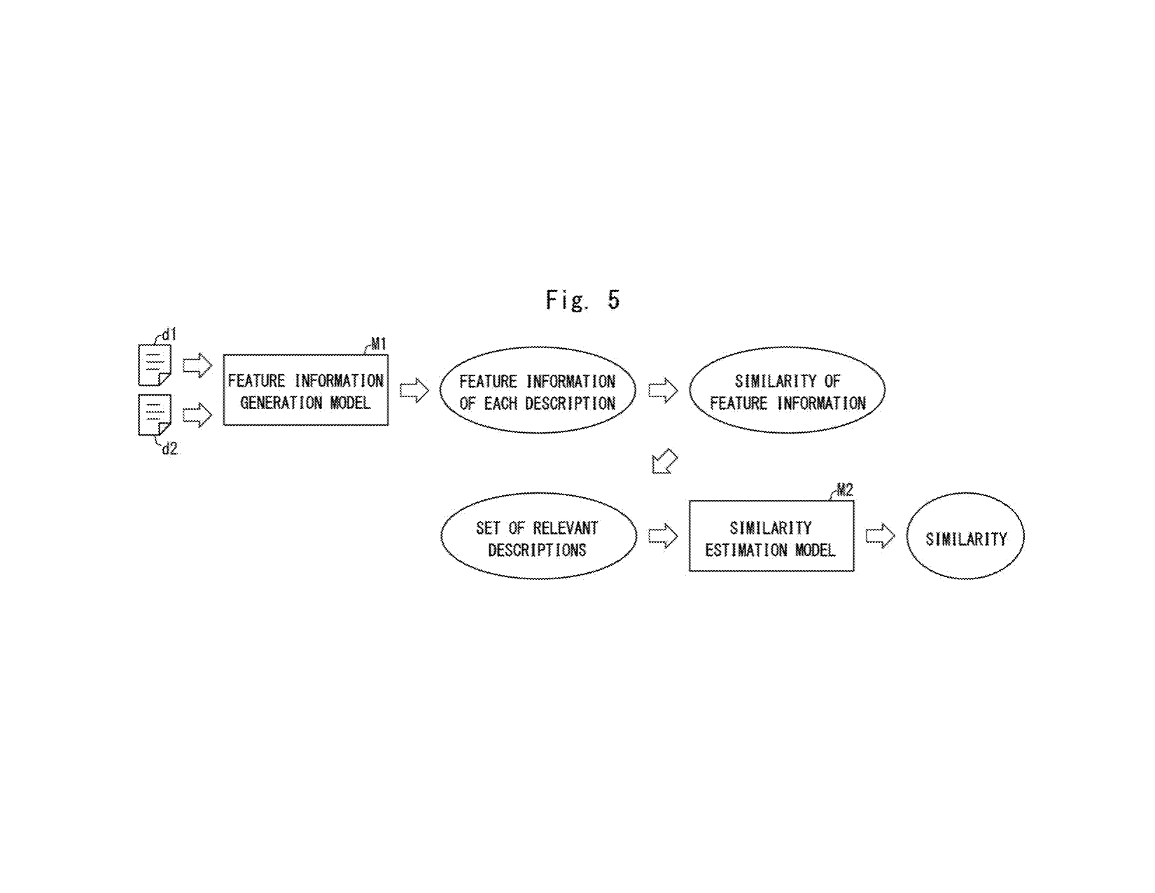

Although details will be described later with reference to FIG. 5, the identification unit 101A uses the feature information generation model M1 and the similarity estimation model M2 in combination to identify a description whose contents relevant to each other between the original data d1 and the medical document d2, more specifically, a description whose contents are similar to each other. As described above, since the feature information generation model M1 and the similarity estimation model M2 are used in combination, the feature information generation model M1 and the similarity estimation model M2 may be integrally configured as one model.

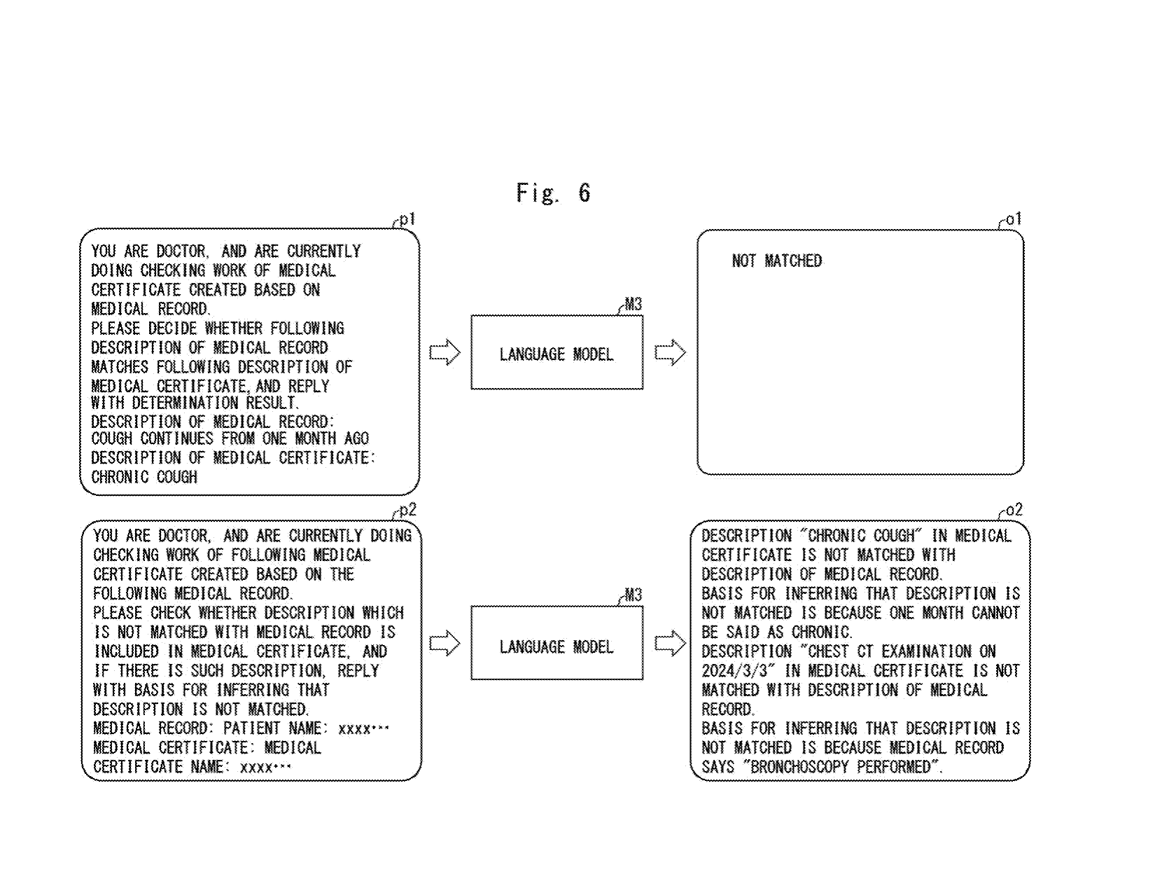

As described above, the language model M3 is a machine-learned model obtained by machine-learning a natural language. The matching determination unit 105A determines whether the contents of the set of descriptions identified by the identification unit 101A match using the language model M3. Determination of consistency using the language model M3 will be described with reference to FIG. 6.

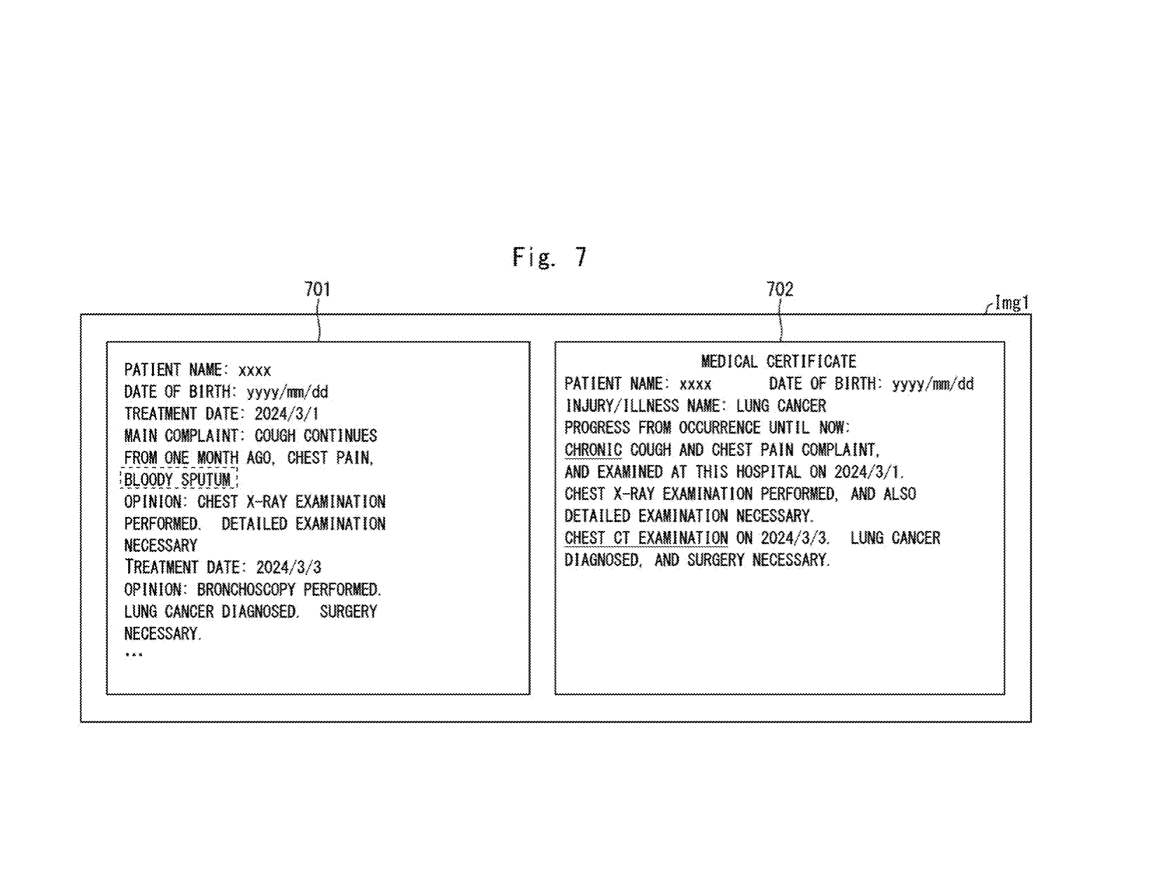

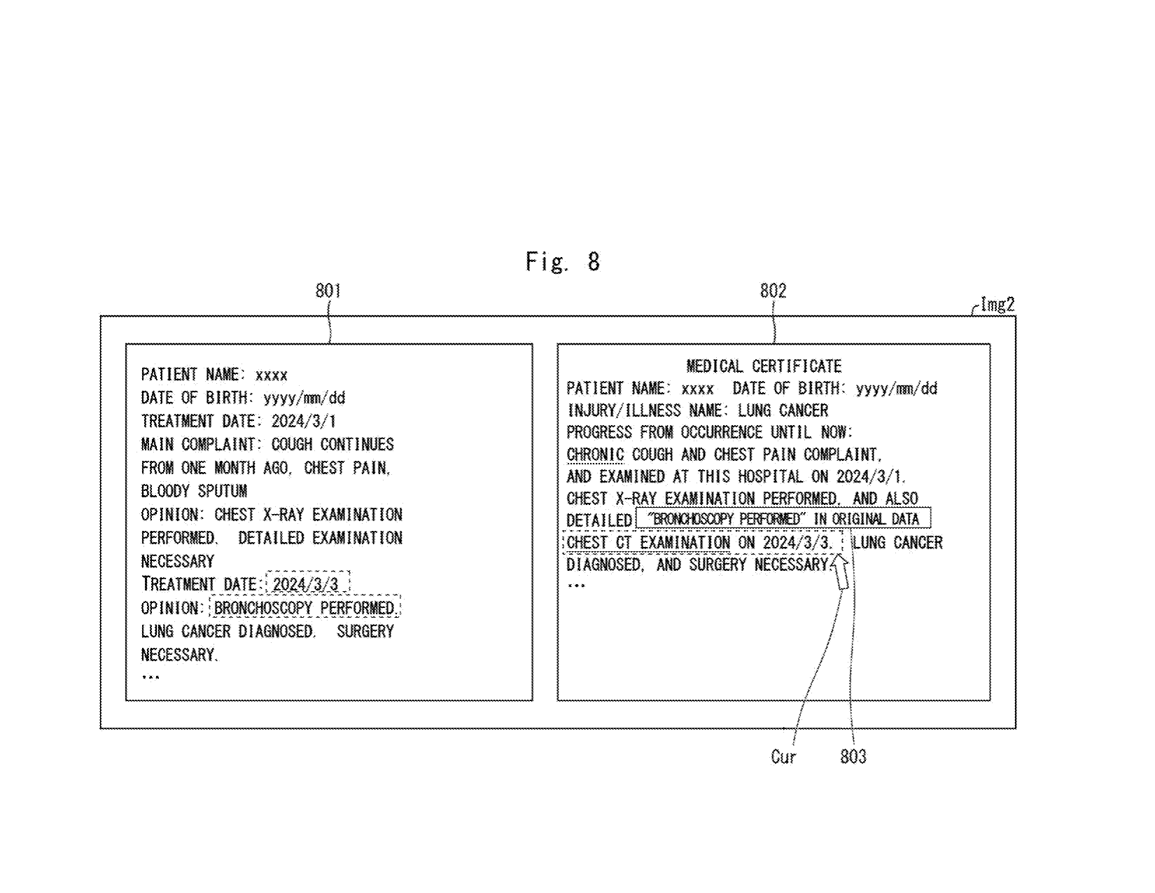

Then, the output control unit 107A presents the results of the determination and detection by the identification unit 101A, the detection unit 102A, and the matching determination unit 105A to the user. For example, the output control unit 107A may display an image indicating the determination and detection results. The image displayed by the output control unit 107A will be described later with reference to FIGS. 7 and 8.

### Method of identifying description of relevant contents

A method of identifying a description of relevant contents using the feature information generation model M1 and the similarity estimation model M2 will be described with reference to FIG. 5. FIG. 5 is a diagram for explaining a method of identifying a description of relevant contents using the feature information generation model M1 and the similarity estimation model M2. As described above, the identification of the description of relevant contents is performed by the identification unit 101A.

As illustrated, the identification unit 101A inputs the original data d1 and the medical document d2 to the feature information generation model M1. At this time, the identification unit 101A separates and divides the original data d1 into each group of contents, and inputs each section obtained by the division to the feature information generation model M1. For example, the identification unit 101A may divide a sentence included in the original data d1 into a plurality of sentences by separating the sentence by a period or the like, and input each sentence obtained by the division to the feature information generation model M1. The same applies to the medical document d2. As a result, for a description (for example, sentence) of each group of contents in the original data d1 and the medical document d2, the feature information indicating the feature of the description is output from the feature information generation model M1.

Next, the identification unit 101A calculates the similarity of the feature information generated by the feature information generation model M1. More specifically, the identification unit 101A performs a process of calculating a similarity between one piece of feature information generated from the original data d1 and one piece of feature information generated from the medical document d2 for each combination of a plurality of pieces of feature information generated from the original data d1 and the medical document d2. For example, in a case where 100 pieces of feature information and 90 pieces of feature information are generated from the original data d1 and the medical document d2, the similarity is calculated for 100 × 90 = 9000 combinations of feature information. A method of calculating the similarity of the feature information is arbitrary. For example, in a case where the feature information is represented by a vector, the identification unit 101A may calculate the cosine similarity.

Next, the identification unit 101A sets descriptions of relevant contents in the original data d1 and the medical document d2 based on the calculated similarity as a set. For example, the identification unit 101A may set one description included in the medical document d2 and a description of the original data d1 relevant to a predetermined number of pieces of feature information (feature information of the original data d1) having a high degree of similarity to the feature information of the description as a set. In a case where the predetermined number is 3, three sets are generated for one description included in the medical document d2. One description included in the original data d1 and a description of the medical document d2 relevant to a predetermined number of pieces of feature information (feature information of the medical document d2) having a high degree of similarity to the feature information of the description may be paired as a set. It can also be said that these processes are a process of selecting a set of descriptions having a high degree of similarity of the feature information or a process of extracting a description candidate of relevant contents.

Next, the identification unit 101A inputs a set of the description of the original data d1 and the description of the medical document d2 generated as described above to the similarity estimation model M2. As a result, the similarity estimation model M2 outputs the similarity of the input description. Then, the identification unit 101A identifies the description of relevant contents based on the output similarity. For example, in a case where three sets are generated for one description included in the medical document d2, the identification unit 101A may identify a description of a set in which the calculated similarity is equal to or greater than a predetermined threshold among the three sets as a description of relevant contents. In this case, a plurality of descriptions may be identified as descriptions of relevant contents. The identification unit 101A may identify a description of a set having the maximum similarity among a plurality of sets of descriptions generated for one description included in the medical document d2 as a description of relevant contents.

The identification unit 101A may identify a description of relevant contents using the similarity estimation model M2 without using the feature information generation model M1. In this case, the identification unit 101A may separate and divide each of the original data d1 and the medical document d2 into groups of the contents, and input the descriptions of the sections obtained by the division into a set to the similarity estimation model M2. For example, in a case where ten and nine descriptions are obtained from the original data d1 and the medical document d2, the identification unit 101A may input 10 × 9 = 90 sets of descriptions to the similarity estimation model M2.

However, in general, the process of calculating the similarity between individual descriptions by the similarity estimation model M2 is more accurate but takes more time than the process of calculating the similarity between the feature information generated by generating the feature information by the feature information generation model M1. Therefore, as in the example of FIG. 5, a set of descriptions to be input to the similarity estimation model M2 may be narrowed down first by processing using the feature information generation model M1, and then a description of relevant contents may be identified by the similarity estimation model M2.

As described above, the identification unit 101A may identify the description of relevant contents using the similarity estimation model M2 that is machine-learned so as to output the similarity of contents of the sentence with respect to a set of input sentences. As a result, in addition to the effect obtained by the information processing device 1, it is possible to identify the description of relevant contents with high accuracy.

As described above, the identification unit 101A may generate the feature information of each description included in the original data d1 and the medical document d2 using the feature information generation model M1 that is machine-learned so as to generate the feature information indicating the feature of the input sentence, and input a set of sentences selected based on the similarity of each piece of the generated feature information to the similarity estimation model M2. As a result, in addition to the effects obtained by the information processing device 1, it is possible to achieve both the processing speed and the accuracy in identifying the description relevant to the content.

### Method of determining consistency

A method of determining consistency using the language model M3 will be described with reference to FIG. 6. FIG. 6 is a diagram for explaining a method of determining consistency using the language model M3. FIG. 6 illustrates an example of using a prompt p1 and an example of using a prompt p2.

The prompt p1 is a prompt for instructing to determine consistency between a set of descriptions identified by the identification unit 101A, that is, descriptions whose contents are identified as relevant by the identification unit 101A. Specifically, in the items of “description of medical record” and “description of medical certificate” in the prompt p1, a set of description of original data “cough continues from one month ago” and description of medical document “chronic cough”, which are identified by the identification unit 101A, is input. The prompt p1 includes a sentence “Please judge whether the following description of the medical record matches the following description of the medical certificate, and answer the judgment result.” instructing to determine consistency between the contents of the above two descriptions.

The prompt p1 includes a sentence “You are a doctor, and currently doing checking work of medical certificate created based on medical record.”. It is not essential to include such a sentence, but the inference accuracy can be expected to be improved by including such a sentence. The expression in the prompt p1 can be appropriately changed within a range in which a desired inference result can be obtained. For example, the matching determination unit 105A may generate a prompt in which the expression of the inference instruction is different according to the type of the target medical document, the type of the target original data, the language model M3 to be used, and the like.

The matching determination unit 105A may generate a prompt that includes an answer format and instructs to answer in the answer format. In this way, it is possible to obtain an inference result in a desired format by designating the answer format. For example, the matching determination unit 105A may generate a prompt including a sentence instructing to answer with either “matched ” or “not matched”. As a result, answers other than “matched” and “not matched” are not output from the language model M3.

In the prompt p1, contents other than “description of medical record” and “description of medical certificate” are fixed. For this reason, a portion other than the contents of “description of medical record” and “description of medical certificate” in the prompt p1 may be stored in the storage unit 11A or the like as a fixed template. As a result, the matching determination unit 105A can generate the prompt p1 by inputting, to the template, the description of the content relevant to the set of descriptions identified by the identification unit 101A, that is, the original data and the medical document.

The matching determination unit 105A inputs the prompt p1 generated as described above to the language model M3. As a result, the inference result regarding the consistency of the set of descriptions identified by the identification unit 101A is output from the language model M3. In the example of FIG. 6, output data o1 indicating the inference result of inconsistency is output from the language model M3.

In what form the inference result is output can be designated by a prompt. For example, the matching determination unit 105A may generate a prompt for instructing to answer with three choices of matching, neutrality, and non-matching. The neutrality in this prompt means that it is neither matching nor non-matching. What kind of processing is to be performed in a case where a neutral answer is output may be determined in advance. For example, the output control unit 107A may present, to the user, a set of descriptions from which a neutral answer has been output, and may cause the user to input whether the descriptions match each other. For example, the matching determination unit 105A may generate a prompt to instruct so as to output a numerical value (for example, a numerical value of 0 to 1) indicating the degree of possibility that the contents of the set of descriptions match. In this case, in a case where the output numerical value is equal to or larger than the predetermined threshold, the matching determination unit 105A may determine that the descriptions of the set match.

As described above, the matching determination unit 105A may generate a prompt for instructing to determine consistency of a set of descriptions identified by the identification unit 101A, and determine whether the contents of the descriptions match based on the output obtained by inputting the generated prompt to the language model M3. As a result, in addition to the effect obtained by the information processing device 1, it is possible to obtain an accurate inference result regarding the consistency in contents of the description.

The matching determination unit 105A can also determine consistency between the description of the original data and the description of the medical document without using a specific result by the identification unit 101A. The prompt p2 illustrated in FIG. 6 is an example of a prompt for determining consistency between the description of the original data and the description of the medical document without using a specific result by the identification unit 101A.

The prompt p2 has substantially the same content as the prompt p1, but is different from the prompt p1 in that the prompt p2 does not include the descriptions extracted from the medical record and the medical certificate, that is, the original data and the medical document, but includes the entire text thereof. The prompt p2 is different from the prompt p1 in that the prompt p2 instructs to answer a description that is not matched together with the basis that the description is not matched. The prompt p2 can be generated by inputting the original data and the medical document to a predetermined template. As described above, “neutrality” may be included as a candidate for output in the prompt. In this case, the prompt may include a sentence instructing to answer the basis even in a case where it is inferred to be neutral.

By inputting the above prompt p2 to the language model M3, an inference result as to whether a description not matching with the original data is included in the medical document is output from the language model M3. In the example of FIG. 6, output data o2 is output from the language model M3. In the output data o2, descriptions that do not match the description of the medical record in the medical certificate are listed. Each of the listed descriptions indicates a basis for inferring that the description is not matched. Although details will be described later, the output control unit 107A may present the above-described basis output by the language model M3 to the user as a determination material for determining the validity of the inference result.

### Repetition of inference

Since the language model M3 is a probabilistic model, inference results in a plurality of inferences can be different from each other even in a case where exactly the same prompt is input. In particular, it is known that there is a tendency that an inference result different from the fact is hardly repeatedly output. Therefore, the matching determination unit 105A may perform processing of inputting a prompt to the language model M3 and outputting the inference result a plurality of times. In this case, the matching determination unit 105A may determine that the contents of a set of descriptions having a large variation in the inference result are not matched. For example, the matching determination unit 105A may calculate a score (for example, a ratio of inference results having different contents from other inference results to all the inference results) indicating the magnitude of the variation in the inference result for each set of descriptions, and determine that a set of descriptions of which the calculated score exceeds a predetermined threshold is not matched in contents.

### First display screen example

An example of a display screen displayed by the output control unit 107A will be described with reference to FIG. 7. FIG. 7 is a diagram illustrating an example of the display screen displayed by the output control unit 107A. The screen example Img1 illustrated in FIG. 7 includes a display area 701 for displaying original data and a display area 702 for displaying a medical document. In the screen example Img1, the detection result of the detection unit 102A is illustrated on the original data illustrated in the display area 701, and the determination result of the matching determination unit 105A is illustrated on the medical document illustrated in the display area 702.

Specifically, in the original data shown in the display area 701, the word “bloody sputum” is marked, so that the word can be distinguished from other descriptions. This indicates that the detection unit 102A has detected the word “bloody sputum”, that is, a description of contents relevant to the word “bloody sputum” has not been identified from the medical document. The word “bloody sputum” is a description that is included in the original data but is not included in the medical document and is suspected of omission of reflection in the medical document.

In the medical document shown in the display area 702, the description “chronic” and the description “chest CT examination” are underlined, so that these descriptions can be distinguished from other descriptions. This indicates that the matching determination unit 105A determines that these descriptions are not matched with the description of the original data.

In this manner, the output control unit 107A may display both the original data and the medical document, and display the detection result of the detection unit 102A and the determination result of the matching determination unit 105A on the displayed original data and medical document. As a result, the user can smoothly confirm the appropriateness/inappropriateness of the content of the medical document and smoothly perform the revision work while comparing the original data with the medical document.

A mode of displaying the detection result of the detection unit 102A and the determination result of the matching determination unit 105A is arbitrary, and is not limited to the example of FIG. 7. For example, the output control unit 107A may highlight the detection result of the detection unit 102A and the determination result of the matching determination unit 105A on the original data and the medical document in a mode different from the example of FIG. 7 (for example, by changing the display color and/or font of characters). As described above, a display mode for making a certain description distinguishable from other descriptions is arbitrary. The same applies to the example of FIG. 8 described later. For example, the output control unit 107A may display the detection result of the detection unit 102A and the determination result of the matching determination unit 105A in a display area different from the display areas 701 and 702, a different screen, or the like.

### Second display screen example

The output control unit 107A may display the identification result of the identification unit 101A, or may display the basis of the determination by the matching determination unit 105A. This will be described with reference to FIG. 8. FIG. 8 is a diagram illustrating another example of the display screen displayed by the output control unit 107A. The screen example Img2 illustrated in FIG. 8 includes a display area 801 for displaying original data and a display area 802 for displaying a medical document.

In the screen example Img2, a part of the description of the medical document displayed in the display area 802 is designated by a cursor Cur. The designated description is marked so that the description can be distinguished from other descriptions. In the display area 801, a description of the original data relevant to the above description designated by the cursor Cur is marked similarly to the designated description, so that the description can be distinguished from other descriptions.

Specifically, a description of “chest CT examination was performed on 2024/3/3” among the descriptions of the medical document displayed in the display area 802 is designated by the cursor Cur. The operation of designating the description is received by the reception unit 106A. Then, since the above description is designated, the description in the display area 802 is highlighted by marking. Due to the designation, the descriptions of “2024/3/3” and “Bronchoscopy performed” among the descriptions of the original data displayed in the display area 801 are also highlighted by marking. These highlighted descriptions are descriptions whose contents have been identified to be relevant by the identification unit 101A.

In this manner, the output control unit 107A may display both the medical document and the original data, and in response to the operation of designating a description of a part of the displayed medical document, display a description of contents relevant to the designated description in a distinguishable manner from other descriptions. As a result, in addition to the effect obtained by the information processing device 1, it is possible to smoothly perform an operation of checking consistency by matching the description of the medical document with the description of the original data.

The description designated in the example of FIG. 8 includes an underlined description, that is, a description determined by the matching determination unit 105A to be not matched with the description of the original data (specifically, description of “chest CT examination”). In this case, as illustrated in the drawing, the output control unit 107A may display basis information 803 indicating the basis that the description is inferred to be not matched with the description of the original data in association with the designated description. As described with reference to FIG. 6, the matching determination unit 105A can cause the language model M3 to output the basis of inference regarding consistency. Therefore, the output control unit 107A may display the basis output by the language model M3 as the basis information 803.

The reception unit 106A may also receive an operation of designating a description of a part of the original data. In this case, the output control unit 107A may display the description of the medical document having the content relevant to the designated description in a distinguishable manner from other descriptions in response to the operation of designating a description of a part of the displayed original data. In a case where it is determined that the designated description (or a part of the description) in the original data does not match the description of the medical document, the output control unit 107A may display the basis information indicating the basis.

As described above, the matching determination unit 105A may cause the language model M3 to output the basis for the consistency determination. Then, the output control unit 107A may cause the basis to be displayed in response to the operation of designating the description determined to be not matched by the matching determination unit 105A. As a result, in addition to the effect obtained by the information processing device 1, it is possible to cause the user to confirm whether the descriptions of the original data and the medical document are matched with reference to the displayed basis. As described above, the result of the consistency determination may include “neutrality”, and it is also possible to cause the language model M3 to output the basis for the determination as being neutral. Therefore, the output control unit 107A may display the basis for the determination as being neutral in response to the operation of designating the description determined as being neutral.

### Flow of processing: overall

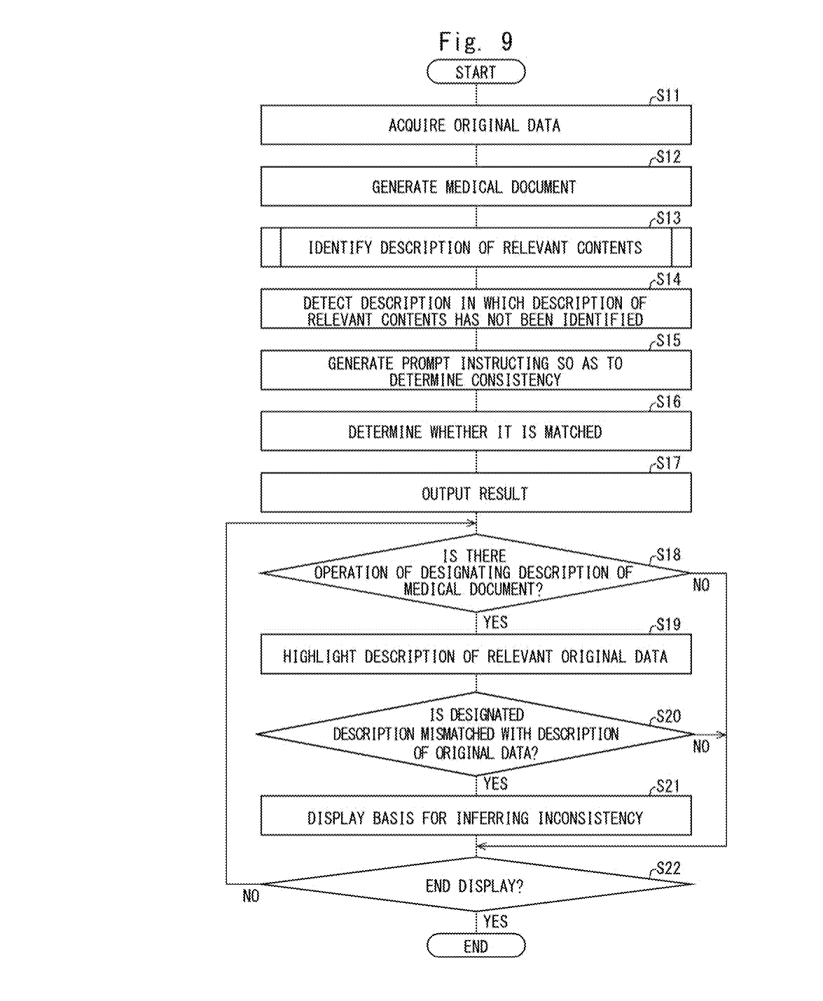

A flow of processing executed by the information processing device 1A will be described with reference to FIG. 9. FIG. 9 is a flowchart illustrating a flow of processing executed by the information processing device 1A. The flowchart of FIG. 9 includes each processing of the confirmation support method according to the present exemplary example embodiment.

In S11, the acquisition unit 103A acquires original data that is data to be a source for generating a medical document. Subsequently, in S12, the document generation unit 104A generates a medical document from the original data acquired in S11.

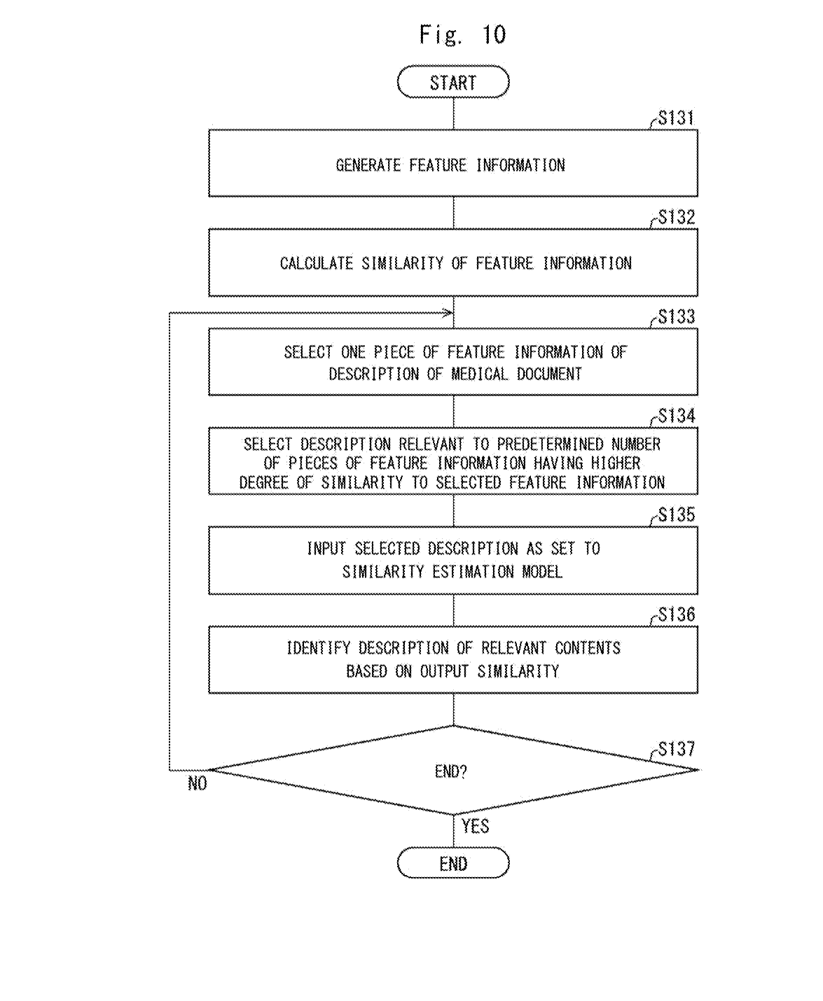

In S13 (identification process), the identification unit 101A identifies descriptions of relevant contents between the original data acquired in S11 and the medical document generated in S12 as targets. Details of the processing of S13 will be described later with reference to FIG. 10.

In S14 (detection process), the detection unit 102A detects a description in which a description of relevant contents has not been identified in S13. In S14, the detection unit 102A may detect a description in which the description of relevant contents is not identified in the medical document among the descriptions included in the original data, that is, a description suspected of omitting reflection in the medical document. In S14, the detection unit 102A may detect, among the descriptions included in the medical document, a description whose relevant contents have not been identified in the original data, that is, a description suspected to have been erroneously written in the medical document. The detection unit 102A may detect both a description suspected to be omitted from the medical document and a description suspected to be erroneously written in the medical document.

In S15, the matching determination unit 105A generates a prompt for instructing to determine consistency of the set of descriptions (set of relevant descriptions) identified in S13. For example, the matching determination unit 105A may generate a prompt such as the prompt p1 in FIG. 6 including the set of descriptions identified in S13. The matching determination unit 105A may generate a prompt such as the prompt p2 in FIG. 6, for example, without using the identification result of S13.

In S16, the matching determination unit 105A determines whether the contents of the description match between the original data and the medical document based on the output obtained by inputting the prompt generated in S15 to the language model M3.

In S17, the output control unit 107A outputs the detection result (suspected omission of reflection in medical document and/or suspected erroneously written in medical document) in S14 and the determination result (description determined not to be matched) in S16. For example, the output control unit 107A may display the medical document and the original data, and display the above-described detection result and the above-described determination result to be displayed on the displayed medical document and the original data, as in the screen example Img1 of FIG. 7. The output control unit 107A may also display a description determined to be neutral by the matching determination unit 105A as a determination result.

In S18, the reception unit 106A determines whether an operation of designating the description of the medical document has been performed. The operation of designating the description may be performed by a cursor as in the example of FIG. 8 using an input device such as a mouse, or may be performed using another input device such as a keyboard or a touch panel. If YES is determined in S18, the process proceeds to S19, and if NO is determined in S18, the process proceeds to S22.

In S19, the output control unit 107A highlights the description of the original data relevant to the designated description. The highlighting may be performed in a display mode in which a target description can be distinguished from other descriptions.

In S20, the output control unit 107A determines whether the designated description includes a description mismatched with the description of the original data. Specifically, the output control unit 107A determines whether the designated description includes a description determined to be not matched in S16. If YES is determined in S20, the process proceeds to S21, and if NO is determined in S20, the process proceeds to S22.

In S21, the output control unit 107A displays the basis for inferring that the designated description is mismatched with the description of the original data. This basis can be output to the language model M3 as described above. For example, as in the example of FIG. 8, the output control unit 107A may display the basis information indicating the basis in association with the designated description.

In S22, the output control unit 107A determines whether to end the display. In S22, for example, in a case where the reception unit 106A receives a predetermined operation for ending the display, it is determined to end the display. In a case where YES is determined in S22, the processing in FIG. 10 is ended. On the other hand, if NO is determined in S22, the processing returns to S18.

As described in the first exemplary example embodiment, in addition to the description that has been erroneously written and the description that has not been reflected, the relevant description may not be detected in a description whose content has been modified to an extent that it cannot be identified that the relevant description has been made, or a description that is unnecessary to be described in a medical document such as a greeting sentence or an acknowledgement. Therefore, the detection unit 102A may detect the remaining description from the description in which the description of relevant contents has not been identified by the identification unit 101A except for at least one of the description in which the content is modified and the description unnecessary to be described in the medical document.

For example, in a case where the description whose content has been modified is excluded, before performing the processing of S14, the matching determination unit 105A may generate a prompt for determining consistency between the description of the original data and the description of the medical document, such as the prompt p2 of FIG. 6, by using the original data acquired in S11 and the medical document generated in S12. Then, the matching determination unit 105A inputs the generated prompt to the language model M3, and detects a description not matching the description of the original data among the descriptions of the medical document based on the output of the language model M3. With this processing, it is possible to detect a description whose content has been modified.

Then, in this case, in S14, the detection unit 102A detects a description in which the description of relevant contents has not been identified in S13, and detects the remaining description obtained by removing the description detected by the matching determination unit 105A as described above from the detected description.

As described above, the detection unit 102A may detect the remaining descriptions from the descriptions of the medical document except for the description identified by the identification unit 101A (that is, the description including the description of the content relevant to the original data) and the description detected by the matching determination unit 105A (that is, the description not matching with the description of the original data). As a result, it is possible to detect only descriptions having a high possibility of reflection omission.

### Flow of processing: S13

Details of the processing of S13 of FIG. 9 will be described with reference to FIG. 10. FIG. 10 is a flowchart illustrating details of the processing of S13 of FIG. 9.

In S131, the identification unit 101A separates and divides the original data acquired in S11 of FIG. 9 into each group of the contents, and inputs each section obtained by the division to the feature information generation model M1 to generate the feature information of each section. The identification unit 101A also separates and divides the medical document generated in S12 of FIG. 9 into each group of the contents similarly to the original data, and inputs each section obtained by the division to the feature information generation model M1 to generate the feature information of each section.

In S132, the identification unit 101A calculates the similarity of the feature information generated in S131. More specifically, the identification unit 101A performs a process of calculating a similarity between one piece of feature information generated from the original data and one piece of feature information generated from the medical document for each combination of a plurality of pieces of feature information generated from the original data and the medical document.

In S133, the identification unit 101A selects one piece of feature information of the description of the medical document from the feature information generated in S131. Next, in S134, the identification unit 101A identifies a predetermined number of pieces of feature information having a high degree of similarity (calculated in S132) to the feature information selected in S133 among the feature information of the original data generated in S131, and selects a description (description of the original data) relevant to each piece of the identified feature information.

In S135, the identification unit 101A sets the description selected in S133 and one of the descriptions selected in S134 as a set and inputs the set to the similarity estimation model M2. This process is performed for each of the descriptions selected in S134. For example, in a case where three descriptions are selected in S134, three sets of descriptions are input to the similarity estimation model M2 in S135, and the similarity for each set is output from the similarity estimation model M2.

In S136, the identification unit 101A identifies the description of the original data having the content relevant to the description of the medical document (the description of the medical document relevant to the feature information selected in S133) based on the similarity output from the similarity estimation model M2 by the processing in S135. For example, the identification unit 101A may identify, as the description of relevant contents, a set of descriptions in which the similarity output in S135 is equal to or greater than a predetermined threshold. If the description of the medical document relevant to the feature information selected in S133 is erroneously written, the description of relevant contents is not identified in S136.

In S137, the identification unit 101A determines whether to end the process of identifying the description of relevant contents. In S137, the identification unit 101A determines to end the processing in S133 to S136 if the processing has been executed for all the feature information of the description of the medical document generated in S131. If YES is determined in S137, the process of FIG. 10 ends, and if NO is determined in S137, the process returns to S133. In S133 transitioning from S137, the identification unit 101A selects one piece of feature information from unselected feature information among the feature information of the description of the medical document generated in S131.

In S133 of FIG. 10, the feature information of the description of the medical document is selected, but the feature information of the original data may be selected. In this case, in S134, the description of the medical document relevant to a predetermined number of pieces of feature information having a higher degree of similarity to the feature information selected in S133 is selected from the feature information of the medical document generated in S131. Then, in this case, in S136, in a case where the description of the original data relevant to the feature information selected in S133 is not reflected in the medical document, the description of relevant contents is not identified.

### Reference example 1

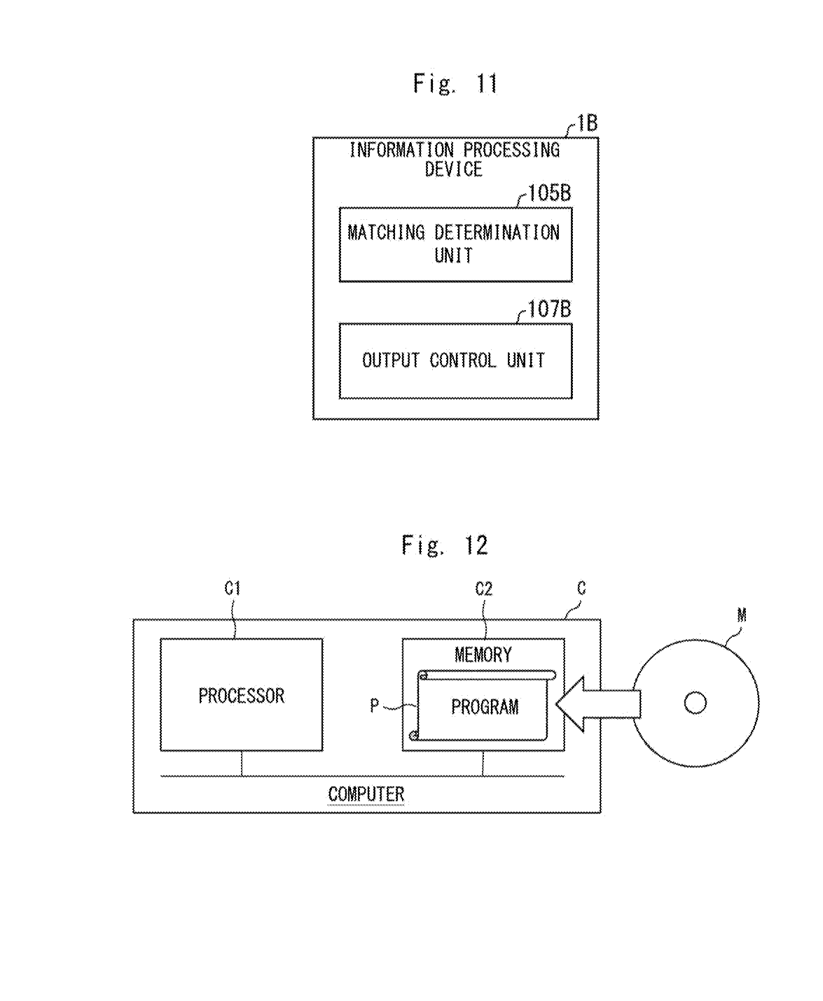

FIG. 11 is a block diagram illustrating a configuration of an information processing device 1B according to the present reference example. As illustrated, the information processing device 1B includes a matching determination unit 105B and an output control unit 107B.

Similarly to the matching determination unit 105A of the second exemplary example embodiment, the matching determination unit 105B determines whether the content of the generated description of the medical document matches the content of the description of the original data, which is data from which the medical document is generated, by using the language model M3 obtained by machine learning of natural language. The medical document to be determined for consistency only needs to be generated by using the original data, and it is similar to the second exemplary example embodiment that a generation subject and a generation method of the medical document are arbitrary. Similarly to the second exemplary example embodiment, the consistency determination may be performed for each set of the description extracted from the medical document and the description extracted from the original data, or may be performed for the entire medical document and the entire original data. In the former case, as in the second exemplary example embodiment, the identification unit 101A and the detection unit 102A may combine descriptions of relevant contents in the medical document and the original data as a set. The identification unit 101A and the detection unit 102A may be included in the information processing device 1B or may be included in another device. In the latter case, the another device may be caused to perform a process of combining descriptions of relevant contents between the medical document and the original data as a set.

The output control unit 107B outputs the determination result of the matching determination unit 105B, similarly to the output control unit 107A of the second exemplary example embodiment. It is similar to the second exemplary example embodiment that what kind of device is caused to output the determination result in what mode is arbitrary.

As described above, the information processing device 1B includes the matching determination unit 105B that determines whether the content of the description of the generated medical document matches the content of the description of the original data, which is data from which the medical document is generated, by using a language model M3 obtained by machine learning of a natural language, and the output control unit 107B that causes a determination result of the matching determination unit 105B to be output. According to the information processing device 1B, it is possible to obtain an effect of facilitating the confirmation work of the medical document.

### Confirmation support program

The above-described functions of the information processing device 1B can also be achieved by a program. A confirmation support program according to the present reference example is a medical document confirmation support program, and causes a computer to function as: a matching determination means for determining whether contents of a description of a generated medical document match contents of a description of original data that is data from which the medical document is generated, using a language model M3 obtained by machine learning of natural language; and an output control means for outputting a determination result of the matching determination means. According to this confirmation support program, it is possible to obtain an effect of facilitating the confirmation work of the medical document.

### Confirmation support method

A confirmation support method according to the present reference example is a medical document confirmation support method, and in the confirmation support method, at least one processor executes a matching determination process of determining whether contents of a generated description of the medical document match contents of a description of original data that is data from which the medical document is generated, using a language model M3 obtained by machine learning of a natural language, and an output control process of outputting a determination result of the matching determination process. According to this confirmation support method, it is possible to obtain an effect of facilitating the confirmation work of the medical document.

### Reference example 2

In the above-described exemplary example embodiment and Reference Example 1, an example has been described in which the confirmation work of the medical document generated from the original data is supported by the information processing devices 1, 1A, and 1B. These information processing devices 1, 1A, and 1B can be used for supporting confirmation work of an arbitrary document in addition to supporting confirmation work of a medical document.

For example, the information processing devices 1, 1A, and 1B can support confirmation work of a design document generated based on a specification. In this case, the specification may be applied instead of the original data in the above-described exemplary example embodiment and Reference Example 1, and the design document generated based on the specification may be applied instead of the medical document. As a result, it is possible to facilitate the confirmation work of the design document conforming to the specification.

For example, the information processing devices 1, 1A, and 1B can also support confirmation work of a summary sentence. In this case, the document to be summarized may be applied instead of the original data in the above-described exemplary example embodiment and Reference Example 1, and the summary sentence summarizing the document may be applied instead of the medical document. This can facilitate the confirmation work of the summary sentence.

### Modified examples

Any execution subject of each processing described in the above-described exemplary example embodiment and reference example is applicable, and is not limited to the above-described examples. For example, a system having functions similar to those of the information processing devices 1, 1A, and 1B can be constructed by a plurality of devices capable of communicating with each other. The executing entity of each process illustrated in the flowcharts of FIGS. 9 and 10 may be one device (also referred to as a processor) or a plurality of devices (also referred to as processors).

### Example of Implementation by Software

Some or all of the functions of the information processing devices 1, 1A, and 1B (referred to below also as “each of the above devices”) may be implemented by hardware such as an integrated circuit (IC chip) or may be implemented by software.

In the latter case, each of the above devices is achieved by, for example, a computer that executes a command of a program as software for achieving each function. An example of such a computer (hereinafter, referred to as a computer C) is illustrated in FIG. 12. FIG. 12 is a block diagram illustrating a hardware configuration of the computer C functioning as each of the above devices.

The computer C includes at least one processor C1 and at least one memory C2. A program (confirmation support program) P for operating the computer C as each of the above devices is recorded in the memory C2. In the computer C, by the processor C1 reading the program P from the memory C2 and executing the program P, each function of each of the above devices is achieved.

As the processor C1, for example, a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination thereof can be used. As the memory C2, for example, a flash memory, a hard disk drive (HDD), a solid state drive (SSD), or a combination thereof can be used.

The computer C may further include a random access memory (RAM) for loading the program P at the time of execution and temporarily storing various types of data. The computer C may further include a communication interface for sending and receiving data to and from another device. The computer C may further include an input/output interface for connecting input/output devices such as a keyboard, a mouse, a display, and a printer.

The program P can be recorded in a non-transitory tangible recording medium M readable by the computer C. As such a recording medium M, for example, a tape, a disk, a card, a semiconductor memory, a programmable logic circuit, or the like can be used. The computer C can acquire the program P via such a recording medium M. The program P can be transmitted via a transmission medium. As such a transmission medium, for example, a communication network, a broadcast wave, or the like can be used. The computer C can also acquire the program P via such a transmission medium.

Each of the above functions of each of the above devices may be achieved by a single processor provided in a single computer, may be achieved in cooperation with a plurality of processors provided in a single computer, or may be achieved in cooperation with a plurality of processors provided in a plurality of computers. The program for causing each of the above devices to achieve each of the above functions may be stored in a single memory provided in a single computer, may be stored in a distributed manner in a plurality of memories provided in a single computer, or may be stored in a distributed manner in a plurality of memories provided in a plurality of computers.

### Supplementary Notes

The present disclosure includes the technologies described in the following Supplementary Notes. However, the present invention is not limited to the technique described in each of Supplementary Notes below, and various modifications can be made within the scope described in the claims.

### Supplementary Note A1

An information processing device including: an identification means for identifying, for original data indicating a medical care history of a patient and a medical document of the patient generated using the original data, a description of relevant contents between the original data and the medical document; and a detection means for detecting a description in which the description of relevant contents has not been identified by the identification means.

### Supplementary Note A2

The information processing device according to Supplementary Note A1, in which the identification means identifies a description of relevant contents by using a similarity estimation model that is machine-learned so as to output similarity of contents of a sentence with respect to a set of input sentences.

### Supplementary Note A3

The information processing device according to Supplementary Note A2, in which the identification means generates feature information of each description included in the original data and the medical document using a feature information generation model that is machine-learned so as to generate feature information indicating a feature of an input sentence, and inputs a set of sentences selected based on a similarity of each piece of the generated feature information to the similarity estimation model.

### Supplementary Note A4

The information processing device according to any one of Supplementary Notes A1 to A3, including a matching determination means for determining whether contents of a set of descriptions identified by the identification means match using a language model obtained by machine learning of a natural language.

### Supplementary Note A5

The information processing device according to Supplementary Note A4, in which the matching determination means generates a prompt for instructing to determine consistency of a set of descriptions identified by the identification means, and determines whether contents of the descriptions match based on an output obtained by inputting the generated prompt to the language model.

### Supplementary Note A6

The information processing device according to Supplementary Note A5, in which the matching determination means causes the language model to output a basis of matching determination, and the information processing device includes an output control means for causing the basis to be displayed in response to an operation of designating a description determined by the matching determination means to be not matched.

### Supplementary Note A7

The information processing device according to any one of Supplementary Notes A1 to A6, including an output control means for displaying both the medical document and the original data, and displaying a description of contents relevant to the designated description in a distinguishable manner from other descriptions in response to an operation of designating a description of a part of the displayed medical document or a description of a part of the displayed original data.

### Supplementary Note A8

An information processing device including: a matching determination means for determining whether contents of a description of a generated medical document match contents of a description of original data, which is data from which the medical document is generated, by using a language model obtained by machine learning of a natural language; and an output control means for outputting a determination result of the matching determination means.

A medical document confirmation support method for causing at least one processor to execute: an identification process of identifying, for original data indicating a medical care history of a patient and a medical document of the patient generated by using the original data, a description of relevant contents between the original data and the medical document; and a detection process of detecting a description in which a description of relevant contents has not been identified in the identification process.

### Supplementary Note B2

The medical document confirmation support method according to Supplementary Note B1, in which in the identification process, the at least one processor identifies a description of relevant contents by using a similarity estimation model that is machine-learned so as to output similarity of contents of a sentence with respect to a set of input sentences.

### Supplementary Note B3

The medical document confirmation support method according to Supplementary Note B2, in which in the identification process, the at least one processor generates feature information of each description included in the original data and the medical document using a feature information generation model that is machine-learned so as to generate feature information indicating a feature of an input sentence, and inputs a set of sentences selected based on a similarity of each piece of the generated feature information to the similarity estimation model.

### Supplementary Note B4

The medical document confirmation support method according to any one of Supplementary Notes B1 to B3, including a matching determination process of determining, by the at least one processor, whether contents of a set of descriptions identified by the identification process match using a language model obtained by machine learning of a natural language.

### Supplementary Note B5

The medical document confirmation support method according to Supplementary Note B4, in which in the matching determination process, the at least one processor generates a prompt for instructing to determine consistency of a set of descriptions identified by the identification process, and determines whether contents of the descriptions match based on an output obtained by inputting the generated prompt to the language model.

### Supplementary Note B6

The medical document confirmation support method according to Supplementary Note B5, including, in the matching determination process, an output control process of outputting, by the at least one processor, a basis of consistency determination to the language model and displaying, by the at least one processor, the basis in response to an operation of designating a description determined to be not matched in the matching determination process.

### Supplementary Note B7

The medical document confirmation support method according to any one of Supplementary Notes B1 to B6, including an output control process of displaying, by the at least one processor, both the medical document and the original data, and displaying a description of contents relevant to the designated description in a distinguishable manner from other descriptions in response to an operation of designating a description of a part of the displayed medical document or a description of a part of the displayed original data.

### Supplementary Note B8

A medical document confirmation support method including: a matching determination process of determining, by at least one processor, whether contents of a description of a generated medical document match contents of a description of original data, which is data from which the medical document is generated, by using a language model obtained by machine learning of a natural language; and an output control process of outputting, by the at least one processor, a determination result of the matching determination process.

### Supplementary Note C1

A medical document confirmation support program for causing a computer to function as an identification means for identifying, for original data indicating a medical care history of a patient and a medical document of the patient generated using the original data, a description of relevant contents between the original data and the medical document, and a detection means for detecting a description in which the description of relevant contents has not been identified by the identification means.

### Supplementary Note C2

The medical document confirmation support program according to Supplementary Note C1, in which the identification means identifies a description of relevant contents by using a similarity estimation model that is machine-learned so as to output similarity of contents of a sentence with respect to a set of input sentences.

### Supplementary Note C3

The medical document confirmation support program according to Supplementary Note C2, in which the identification means generates feature information of each description included in the original data and the medical document using a feature information generation model that is machine-learned so as to generate feature information indicating a feature of an input sentence, and inputs a set of sentences selected based on a similarity of each piece of the generated feature information to the similarity estimation model.

### Supplementary Note C4

The medical document confirmation support program according to any one of Supplementary Notes C1 to C3, in which the program causes the computer to function as a matching determination means for determining whether contents of a set of descriptions identified by the identification means match using a language model obtained by machine learning of a natural language.

### Supplementary Note C5

The medical document confirmation support program according to Supplementary Note C4, in which the matching determination means generates a prompt for instructing to determine consistency of a set of descriptions identified by the identification means, and determines whether contents of the descriptions match based on an output obtained by inputting the generated prompt to the language model.

### Supplementary Note C6

The medical document confirmation support program according to Supplementary Note C5, in which the matching determination means causes the language model to output a basis of matching determination, and the program causes the computer to function as an output control means for causing the basis to be displayed in response to an operation of designating a description determined by the matching determination means to be not matched.

### Supplementary Note C7

The medical document confirmation support program according to any one of Supplementary Notes C1 to C6, in which the program causes the computer to function as an output control means for displaying both the medical document and the original data, and displaying a description of contents relevant to the designated description in a distinguishable manner from other descriptions in response to an operation of designating a description of a part of the displayed medical document or a description of a part of the displayed original data.

### Supplementary Note C8

A medical document confirmation support program for causing a computer to function as a matching determination means for determining whether contents of a description of a generated medical document match contents of a description of original data, which is data from which the medical document is generated, by using a language model obtained by machine learning of a natural language, and an output control means for outputting a determination result of the matching determination means.

### Supplementary Note D1

An information processing device including at least one processor, in which the at least one processor executes an identification process of identifying, for original data indicating a medical care history of a patient and a medical document of the patient generated by using the original data, a description of relevant contents between the original data and the medical document, and a detection process of detecting a description in which a description of relevant contents has not been identified by the identification process.

The information processing device may further include a memory. The memory may store a program for causing the at least one processor to execute each type of the processing.

### Supplementary Note D2

The information processing device according to Supplementary Note D1, in which in the identification process, the at least one processor identifies a description of relevant contents by using a similarity estimation model that is machine-learned so as to output similarity of contents of a sentence with respect to a set of input sentences.

### Supplementary Note D3

The information processing device according to Supplementary Note D2, in which in the identification process, the at least one processor generates feature information of each description included in the original data and the medical document using a feature information generation model that is machine-learned so as to generate feature information indicating a feature of an input sentence, and inputs a set of sentences selected based on a similarity of each piece of the generated feature information to the similarity estimation model.

### Supplementary Note D4

The information processing device according to any one of Supplementary Notes D1 to D3, in which the at least one processor executes a matching determination process of determining whether contents of a set of descriptions identified by the identification process match using a language model obtained by machine learning of a natural language.

### Supplementary Note D5

The information processing device according to Supplementary Note D4, in which in the matching determination process, the at least one processor generates a prompt for instructing to determine consistency of a set of descriptions identified by the identification process, and determines whether contents of the descriptions match based on an output obtained by inputting the generated prompt to the language model.

### Supplementary Note D6

The information processing device according to Supplementary Note D5, in which in the matching determination process, the at least one processor executes an output control process of outputting a basis of consistency determination to the language model and displaying, by the at least one processor, the basis in response to an operation of designating a description determined to be not matched in the matching determination process.

### Supplementary Note D7

The information processing device according to any one of Supplementary Notes D1 to D6, in which the at least one processor executes an output control process of displaying, by the at least one processor, both the medical document and the original data, and displaying a description of contents relevant to the designated description in a distinguishable manner from other descriptions in response to an operation of designating a description of a part of the displayed medical document or a description of a part of the displayed original data.

### Supplementary Note D8

An information processing device including at least one processor, in which the at least one processor executes a matching determination process of determining whether contents of a description of a generated medical document match contents of a description of original data, which is data from which the medical document is generated, by using a language model obtained by machine learning of a natural language, and an output control process of outputting a determination result of the matching determination process.

### Supplementary Note E1

A non-transitory recording medium having stored therein a medical document confirmation support program for causing a computer to execute an identification process of identifying, for original data indicating a medical care history of a patient and a medical document of the patient generated using the original data, a description of relevant contents between the original data and the medical document, and a detection process of detecting a description in which the description of relevant contents has not been identified by the identification process.

### Supplementary Note E2

A non-transitory recording medium having stored therein a medical document confirmation support program for causing a computer to execute a matching determination process of determining whether contents of a description of a generated medical document match contents of a description of original data, which is data from which the medical document is generated, by using a language model obtained by machine learning of a natural language, and an output control process of outputting a determination result of the matching determination process.

1. An information processing device comprising:
at least one memory storing instructions; and
at least one processor configured to execute the instructions to;
identify, for original data indicating a medical care history of a patient and a medical document of the patient generated using the original data, a description of relevant contents between the original data and the medical document; and
detect a description in which the description of relevant contents has not been identified.

2. The information processing device according to claim 1, wherein the at least one processor is further configured to execute the instructions to:
identify a description of relevant contents by using a similarity estimation model that is machine-learned so as to output similarity of contents of a sentence with respect to a set of input sentences.

3. The information processing device according to claim 2, wherein the at least one processor is further configured to execute the instructions to:
generate feature information of each description included in the original data and the medical document using a feature information generation model that is machine-learned so as to generate feature information indicating a feature of an input sentence, and input a set of sentences selected based on a similarity of each piece of the generated feature information to the similarity estimation model.

4. The information processing device according to claim 1, wherein the at least one processor is further configured to execute the instructions to:
determine whether contents of a set of descriptions identified match using a language model obtained by machine learning of a natural language.

5. The information processing device according to claim 4, wherein the at least one processor is further configured to execute the instructions to:
generate a prompt for instructing to determine consistency of a set of descriptions identified, and determine whether contents of the descriptions match based on an output obtained by inputting the generated prompt to the language model.

6. The information processing device according to claim 5, wherein the at least one processor is further configured to:
cause the language model to output a basis for a determination of non-matching content, and
cause the display of the basis in response to a user operation a description determined to have non-matching content.

7. The information processing device according to claim 1, wherein the at least one processor is further configured to execute the instructions to:
display both the medical document and the original data, and display a description of contents relevant to the designated description in a distinguishable manner from other descriptions in response to an operation of designating a description of a part of the displayed medical document or a description of a part of the displayed original data.

8. A medical document confirmation support method for causing at least one processor to execute:
an identification process of identifying, for original data indicating a medical care history of a patient and a medical document of the patient generated by using the original data, a description of relevant contents between the original data and the medical document; and
a detection process of detecting a description in which a description of relevant contents has not been identified in the identification process.

9. A non-transitory storage medium storing an information processing program for causing a computer to function as an information processing apparatus, the program causing the computer to perform processing including:
identification processing of identifying, for original data indicating a medical care history of a patient and a medical document of the patient generated using the original data, a description of relevant contents between the original data and the medical document; and
detection processing of detecting a description in which the description of relevant contents has not been identified.
